(19) United States
(12) Patent Application Publication
(10) Pub. No.: US 2005/0131385 A1
(43) Pub. Date: Jun. 16, 2005

(54) Cannulae for selectively enhancing blood flow

(76) Inventors: Steven F. Bolling, Ann Arbor, MI (US); Anthony Viole, Foothill Ranch, CA (US); Shawn O'Leary, Mission Viejo, CA (US); Robert Pecor, Aliso Viejo, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/735,413

(22) Filed: Dec. 12, 2003

(51) Int. Cl. A61M 29/00

(52) U.S. Cl. 604/509; 604/96.01; 600/16

(57) ABSTRACT

A perfusion cannula system for directing blood through the vasculature of a patient comprises a cannula body having a proximal end, a distal end, and at least one lumen extending therebetween. The perfusion cannula system enhances blood flow past the cannula when the cannula body resides within the patient. A first balloon can be located on an exterior surface of the cannula body and the balloon can be deployed within the vasculature whereby space may be provided between a vessel wall and the cannula body. A cannula body can have an aperture formed therein in fluid communication with a lumen. A sleeve can be carried by the cannula and can be configured to be moveable relative to the aperture to selectively cover and uncover the aperture as desired.

### BACKGROUND OF THE INVENTION

1. Field of the Invention

This application relates to cannulae and, in particular, to cannulae capable of enhancing blood flow around the cannulae within the vasculature of a patient.

2. Description of the Related Art

Treatment and diagnosis of a variety of health conditions in a patient can involve withdrawing blood from the patient's vascular system. For example, a syringe can be inserted into the patient's vasculature to withdraw blood for testing. It is sometimes necessary to introduce blood or other fluids into a patient's vasculature, e.g., an injection via an intravenous line, to provide treatment or obtain a diagnosis.

Treatment of organ failure can involve coordinated withdrawal and introduction of blood, in connection with some additional treatment. Dialysis, for example, involves withdrawing blood from the vasculature, filtering the blood, and infusing the blood back into the vasculature for further circulation. An emerging treatment for congestive heart failure involves coordinated withdrawal of blood from and infusion of blood into the vasculature without further treatment. Both such treatments sometimes call for the insertion of a cannula into the vasculature of the patient.

The size of the cannula employed in these and other vascular treatments can sometimes approach the size of the vessel into which it is inserted. For example, relatively large cannula size may be required where the treatment requires significant amounts of blood to be withdrawn at relatively high flow rates. The desirability of employing multilumen cannulae is another factor that contributes to increased cannula size. Depending on the application, larger cannulae can present a risk to tissue located downstream of where the cannulae are applied. For example, as the size of the cannula to be introduced approaches the size of the blood vessel, blood-flow downstream of the cannula may be restricted. Prolonged restriction of the vessel can lead to ischemia-related pathology.

### SUMMARY OF THE INVENTION

Overcoming many if not all of the limitations of the prior art, the present invention, in one embodiment, provides a perfusion cannula system for directing blood through the vasculature of a patient. The cannula system includes a cannula body that comprises a proximal end, a distal end, and at least one lumen extending therebetween. The cannula system also includes a balloon and a means for deploying the balloon within the vasculature. The balloon is located on an exterior surface of the cannula body. The cannula system provides space between a vessel wall and the cannula body when the cannula body resides within the patient to permit blood flow past the cannula body.

In another embodiment, a perfusion cannula system for directing blood through the vasculature of a patient comprises means for creating space around the cannula body within the vasculature to permit blood flow past the cannula.

In another embodiment, a perfusion system for directing blood through the vasculature of a patient comprises a multilumen cannula. A plurality of radially spaced balloons are configured to be selectively inflated while residing with the vasculature to create space around the cannula within the vasculature to permit blood flow past the cannula.

In an additional embodiment, a perfusion cannula system comprises a cannula body having an aperture formed therein in fluid communication with a lumen. A sleeve is carried by the cannula and is configured to be moveable relative to the aperture to selectively cover and uncover the aperture as desired.

In another embodiment, a perfusion cannula system comprises means for enhancing blood flow past the cannula when the cannula body resides within the patient.

In another embodiment, an extracardiac heart assist system comprises a pump that has an inlet and an outlet. An inflow conduit is coupled with the inlet. An outflow conduit is coupled with the outlet. An intravascular conduit is configured to provide fluid communication between the vasculature of a patient and at least one of the inflow conduit and the outflow conduit. The intravascular conduit has a proximal end, a distal end, at least one lumen extending therebetween, and a means for selectively enhancing blood flow past the cannula when the cannula resides within the patient.

In another embodiment, a method of treating a patient using an extracardiac heart assist system comprises the steps of: inserting a cannula system into the vasculature of a patient, the cannula system being actuatable to enhance blood flow past the cannula when the cannula resides in the vasculature of the patient; and selectively actuating the cannula system, whereby blood flow past the cannula is enhanced.

### BRIEF DESCRIPTION OF THE DRAWINGS

These and other features and advantages of the invention will now be described with reference to the drawings, which are intended to illustrate and not to limit the invention.

FIG. 1 is a schematic view of one embodiment of a heart assist system having multiple conduits for multi-site application, shown applied to a patient's vascular system;

FIG. 2 is a schematic view of another application of the embodiment of FIG. 1;

FIG. 3 is a schematic view of another embodiment of a heart assist system having multiple conduits for multi-site application wherein each of the conduits is applied to more than one vessel, shown applied to a patient's vascular system;

FIG. 4 is a schematic view of another embodiment of a heart assist system having multiple conduits for multi-site application and employing a connector with a T-shaped fitting, shown applied to a patient's vascular system;

FIG. 5 is a schematic view of an L-shaped connector coupled with an inflow conduit, shown inserted within a blood vessel;

FIG. 6 is a schematic view of another embodiment of a heart assist system having multiple conduits for multi-site application, shown applied to a patient's vascular system;

FIG. 7 is a schematic view of another application of the embodiment of FIG. 6, shown applied to a patient's vascular system;

FIG. 8 is a schematic view of another application of the embodiment of FIG. 6, shown applied to a patient's vascular system;

FIG. 9 is a schematic view of another embodiment of a heart assist system having multiple conduits for multi-site application, a reservoir, and a portable housing for carrying a portion of the system directly on the patient;

FIG. 10 is a schematic view of another embodiment of a heart assist system having a multilumen cannula for single-site application, shown applied to a patient's vascular system;

FIG. 11 is a schematic view of a modified embodiment of the heart assist system of FIG. 10, shown applied to a patient's vascular system;

FIG. 12 is a schematic view of another embodiment of a heart assist system having multiple conduits for single-site application, shown applied to a patient's circulatory system;

FIG. 13 is a schematic view of another application of the embodiment of FIG. 12, shown applied to a patient's vascular system;

FIG. 14 is a schematic view of one application of an embodiment of a heart assist system having an intravascular pump enclosed in a protective housing, wherein the intravascular pump is inserted into the patient's vasculature through a non-primary vessel;

FIG. 15 is a schematic view of another embodiment of a heart assist system having an intravascular pump housed within a conduit having an inlet and an outlet, wherein the intravascular pump is inserted into the patient's vasculature through a non-primary vessel;

FIG. 16 is a schematic view of a modified embodiment of the heart assist system of FIG. 15 in which an additional conduit is shown adjacent the conduit housing the pump, and in which the pump comprises a shaft-mounted helical thread;

FIG. 17 is a schematic view of one embodiment of a perfusion cannula system;

FIG. 18 is a schematic view of another embodiment of a perfusion cannula system;

FIG. 19 is a schematic view of another embodiment of a perfusion cannula system;

FIG. 20 is a schematic view of an application to a patient of a heart assist system including a perfusion cannula system according to the embodiment shown in FIG. 17;

FIG. 21 is an enlarged schematic view of a portion of FIG. 20, showing how space may be created by the embodiment shown in FIG. 17;

FIG. 22 is a cross-sectional view of taken along the section plane 22-22 shown in FIG. 21;

FIG. 23 is an enlarged schematic view similar to that of FIG. 21, showing how space may be created by the embodiment shown in FIG. 18;

FIG. 24 is a cross-sectional view of taken along the section plane 24-24 shown in FIG. 23;

FIG. 25 is an enlarged schematic view similar to that of FIG. 21 of the embodiment shown in FIG. 19, which is shown in a first configuration; and

FIG. 26 is an enlarged schematic view showing how space may be created by the embodiment shown in FIG. 19 when in a second configuration.

### DETAILED DESCRIPTION OF THE PREFERRED EMBODIMENT

Turning now to the drawings provided herein, more detailed descriptions of various embodiments of heart assist systems and cannulae for use therewith are provided below.

### I. Extracardiac Heart Assist Systems and Methods

A variety of cannulae are described herein that can be used in connection with a variety of heart assist systems that supplement blood perfusion. Such systems preferably are extracardiac in nature. In other words, the systems supplement blood perfusion, without the need to interface directly with the heart and aorta. Thus, the systems can be applied without major invasive surgery. The systems also lessen the hemodynamic burden or workload on the heart by reducing afterload, impedence, and/or left ventricular end diastolic pressure and volume (preload). The systems also advantageously increase peripheral organ perfusion and provide improvement in neurohormonal status. As discussed more fully below, the systems can be applied using one or more cannulae, one or more vascular grafts, and a combination of one or more cannulae and one or more vascular grafts. For systems employing cannula(e), the cannula(e) can be applied through multiple percutaneous insertion sites (sometimes referred to herein as a multi-site application) or through a single percutaneous insertion site (sometimes referred to herein as a single-site application).

A. Heart Assist Systems and Methods Employing Multi-Site Application

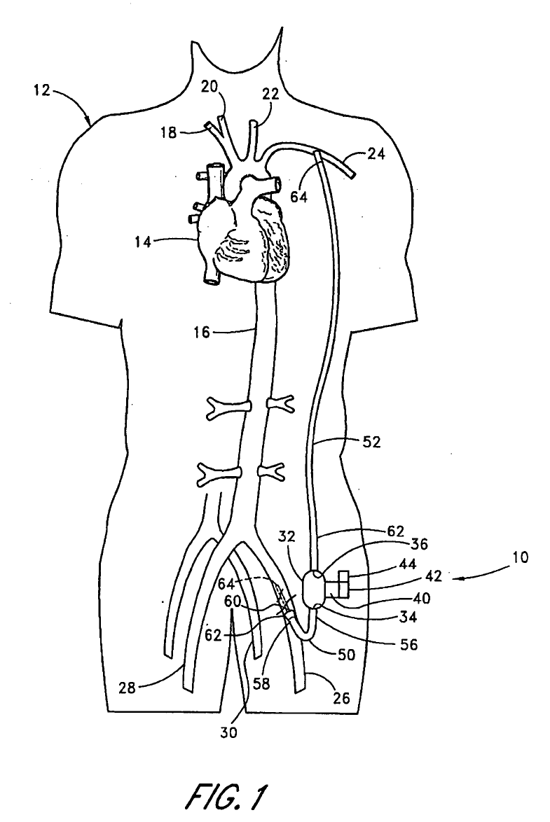

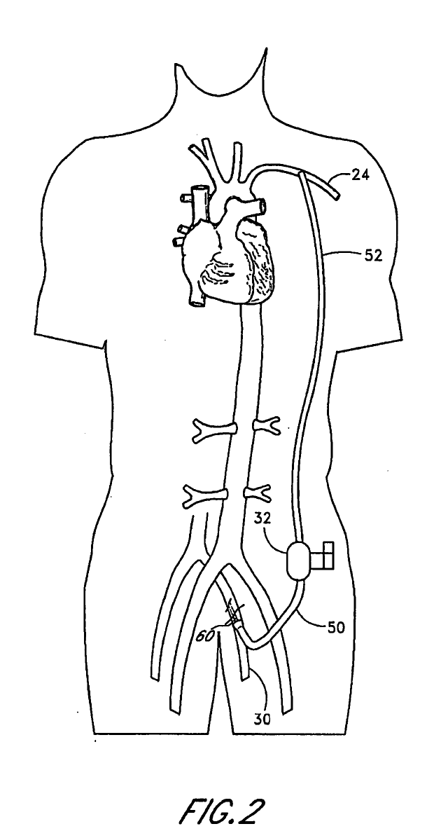

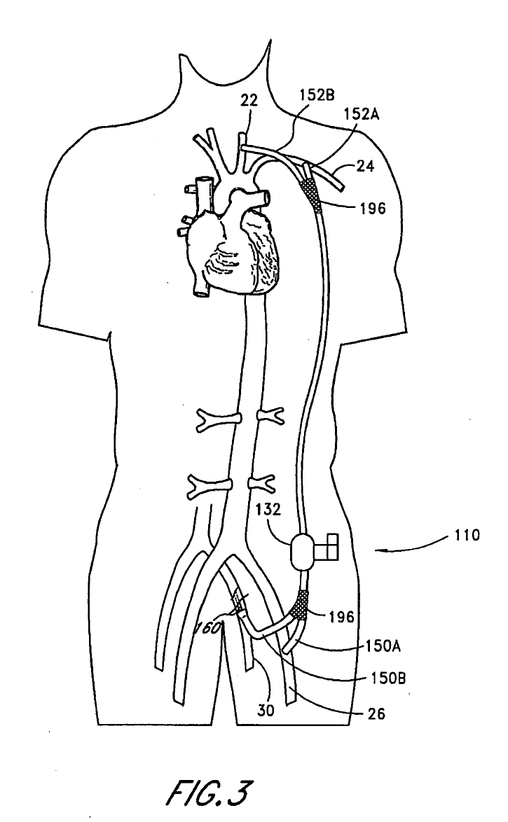

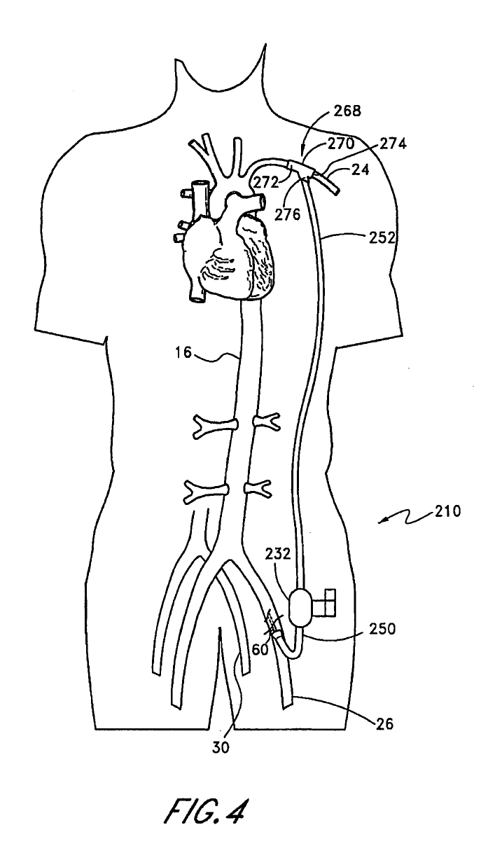

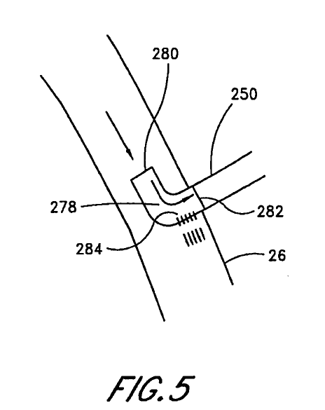

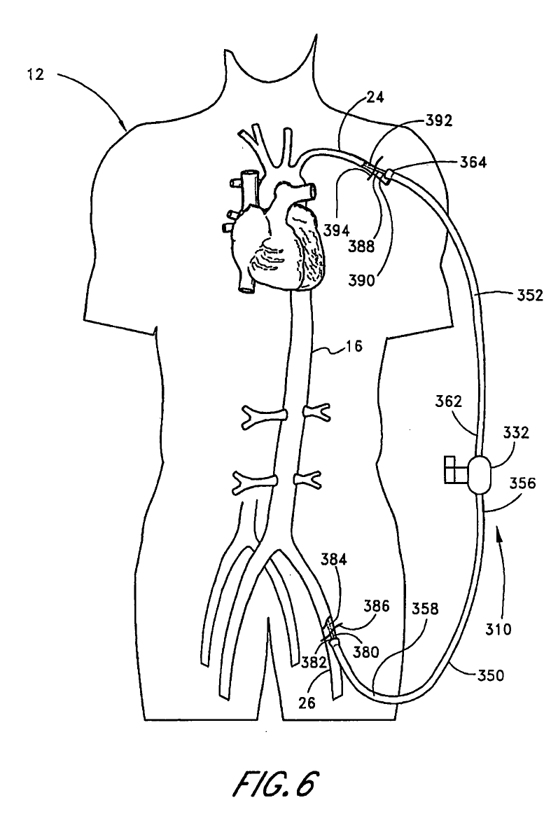

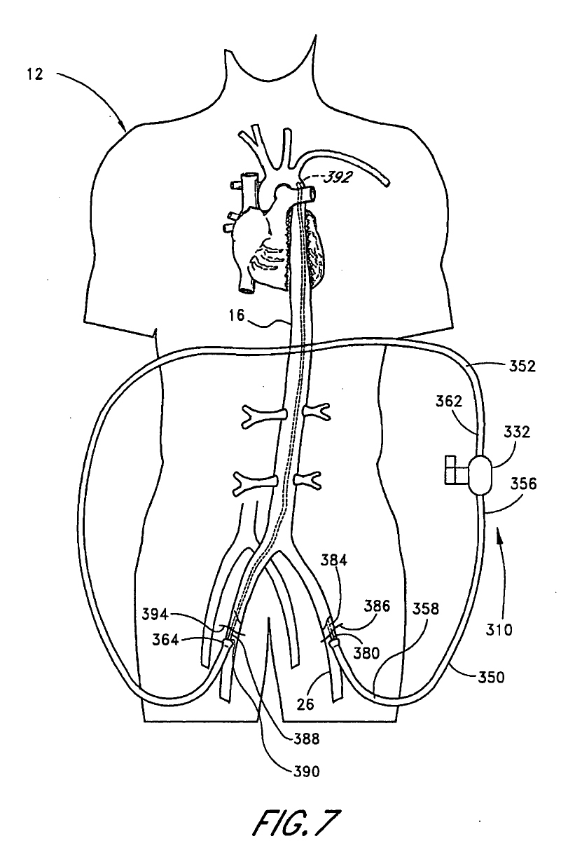

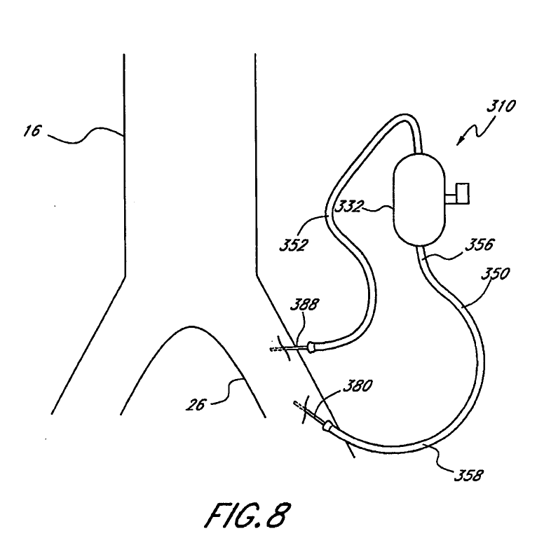

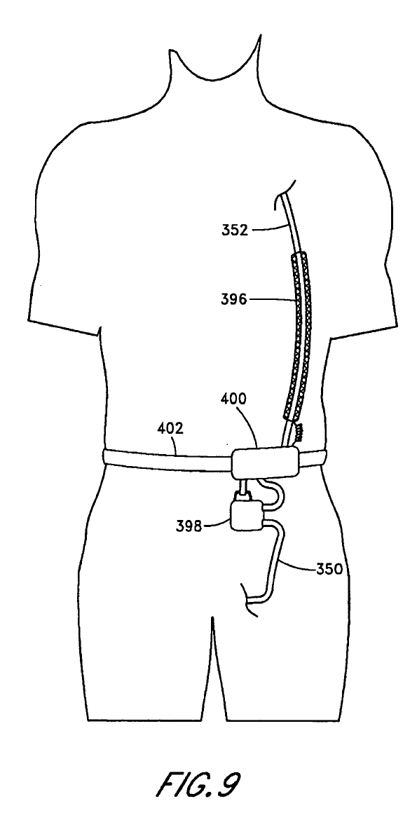

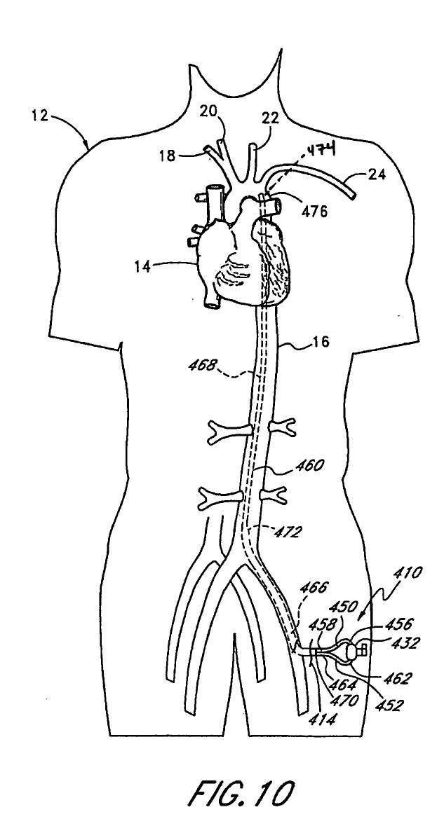

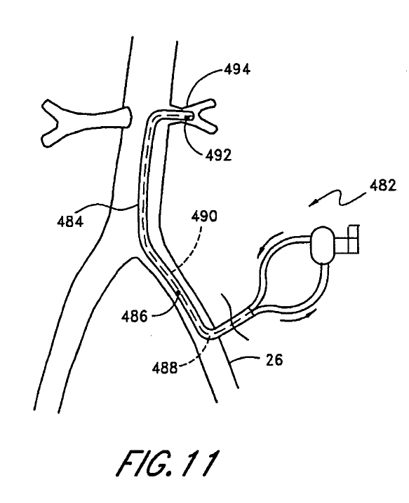

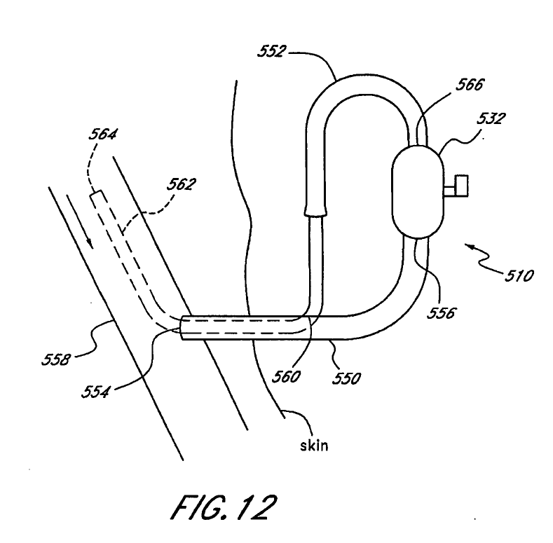

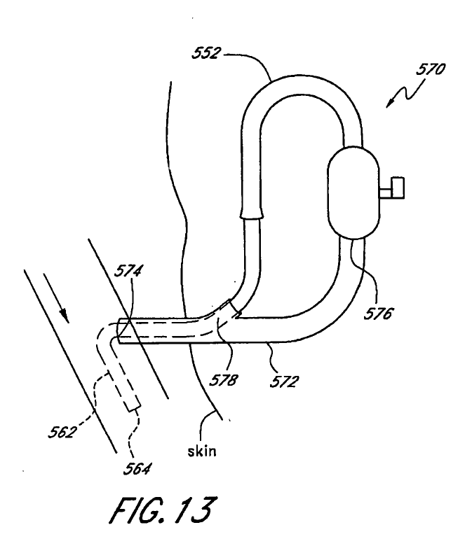

With reference to FIG. 1, a first embodiment of a heart assist system 10 is shown applied to a patient 12 having an ailing heart 14 and an aorta 16, from which peripheral brachiocephalic blood vessels extend, including the right subclavian artery 18, the right carotid artery 20, the left carotid artery 22, and the left subclavian artery 24. Extending from the descending aorta is another set of peripheral blood vessels, the left and right iliac arteries which transition into the left and right femoral arteries 26, 28, respectively. As is known, each of the arteries 16, 18, 20, 22, 24, 26, and 28 generally conveys blood away from the heart. The vasculature includes a venous system that generally conveys blood to the heart. As will be discussed in more detail below, the heart assist systems described herein can also be applied to non-primary veins, including the left femoral vein 30.

The heart assist system 10 comprises a pump 32, having an inlet 34 and an outlet 36 for connection of conduits thereto. The pump 32 preferably is a rotary pump, either an axial type or a centrifugal type, although other types of pumps may be used, whether commercially-available or customized. The pump 32 preferably is sufficiently small to be implanted subcutaneously and preferably extrathoracically, for example in the groin area of the patient 12, without the need for major invasive surgery. Because the heart assist system 10 is an extracardiac system, no valves are necessary. Any inadvertent backflow through the pump 32 and/or through the inflow conduit would not harm the patient 12.

Regardless of the style or nature chosen, the pump 32 is sized to generate blood flow at subcardiac volumetric rates, less than about 50% of the flow rate of an average healthy heart, although flow rates above that may be effective. Thus, the pump 32 is sized and configured to discharge blood at volumetric flow rates anywhere in the range of 0.1 to 3 liters per minute, depending upon the application desired and/or the degree of need for heart assist. For example, for a patient experiencing advanced congestive heart failure, it may be preferable to employ a pump that has an average subcardiac rate of 2.5 to 3 liters per minute. In other patients, particularly those with minimal levels of heart failure, it may be preferable to employ a pump that has an average subcardiac rate of 0.5 liters per minute or less. In yet other patients it may be preferable to employ a pump that is a pressure wave generator that uses pressure to augment the flow of blood generated by the heart.

In one embodiment, the pump 32 is a continuous flow pump, which superimposes continuous blood-flow on the pulsatile aortic blood-flow. In another embodiment, the pump 32 has the capability of synchronous actuation; i.e., it may be actuated in a pulsatile mode, either in copulsating or counterpulsating fashion.

For copulsating action, it is contemplated that the pump 32 would be actuated to discharge blood generally during systole, beginning actuation, for example, during isovolumic contraction before the aortic valve opens or as the aortic valve opens. The pump 32 would be static while the aortic valve is closed following systole, ceasing actuation, for example, when the aortic valve closes.

For counterpulsating actuation, it is contemplated that the pump 32 would be actuated generally during diastole, ceasing actuation, for example, before or during isovolumic contraction. Such an application would permit and/or enhance coronary blood perfusion. In this application, it is contemplated that the pump 32 would be static during the balance of systole after the aortic valve is opened, to lessen the burden against which the heart must pump. The aortic valve being open encompasses the periods of opening and closing, wherein blood is flowing therethrough.

It should be recognized that the designations copulsating and counterpulsating are general identifiers and are not limited to specific points in the patient's heart cycle when the pump 32 begins and discontinues actuation. Rather, they are intended to generally refer to pump actuation in which the pump 32 is actuating, at least in part, during systole and diastole, respectively. For example, it is contemplated that the pump 32 might be activated to be out of phase from true copulsating or counterpulsating actuation described herein, and still be synchronous, depending upon the specific needs of the patient or the desired outcome. One might shift actuation of the pump 32 to begin prior to or after isovolumic contraction or to begin before or after isovolumic relaxation.

Furthermore, the pulsatile pump may be actuated to pulsate asynchronously with the patient's heart. Typically, where the patient's heart is beating irregularly, there may be a desire to pulsate the pump 32 asynchronously so that the perfusion of blood by the heart assist system 10 is more regular and, thus, more effective at oxygenating the organs. Where the patient's heart beats regularly, but weakly, synchronous pulsation of the pump 32 may be preferred.

The pump 32 is driven by a motor 40 and/or other type of drive means and is controlled preferably by a programmable controller 42 that is capable of actuating the pump 32 in pulsatile fashion, where desired, and also of controlling the speed or output of the pump 32. For synchronous control, the patient's heart would preferably be monitored with an EKG in which feedback would be provided the controller 42. The controller 42 is preferably programmed by the use of external means. This may be accomplished, for example, using RF telemetry circuits of the type commonly used within implantable pacemakers and defibrillators. The controller may also be autoregulating to permit automatic regulation of the speed, and/or regulation of the synchronous or asynchronous pulsation of the pump 32, based upon feedback from ambient sensors monitoring parameters, such as pressure or the patient's EKG. It is also contemplated that a reverse-direction pump be utilized, if desired, in which the controller is capable of reversing the direction of either the drive means or the impellers of the pump. Such a pump might be used where it is desirable to have the option of reversing the direction of circulation between two blood vessels.

Power to the motor 40 and the controller 42 may be provided by a power source 44, such as a battery, that is preferably rechargeable by an external induction source (not shown), such as an RF induction coil that may be electromagnetically coupled to the battery to induce a charge therein. Alternative power sources are also possible, including a device that draws energy directly from the patient's body; e. g., the patient's muscles, chemicals or heat. The pump can be temporarily stopped during recharging with no appreciable life threatening effect, because the system only supplements the heart, rather than substituting for the heart.

While the controller 42 and power source 44 are preferably pre-assembled to the pump 32 and implanted therewith, it is also contemplated that the pump 32 and motor 40 be implanted at one location and the controller 42 and the power source 44 be implanted in a separate location. In one alternative arrangement, the pump 32 may be driven externally through a percutaneous drive line or cable, as shown in FIG. 16. In another variation, the pump, motor and controller may be implanted and powered by an extracorporeal power source. In the latter case, the power source could be attached to the side of the patient to permit fully ambulatory movement.

The inlet 34 of the pump 32 is preferably connected to an inflow conduit 50 and an outflow conduit 52 to direct blood flow from one peripheral blood vessel to another. The conduits 50, 52 preferably are flexible conduits, as discussed more fully below. The conduits 50, 52 are coupled with the peripheral vessels in different ways in various embodiments of the heart assist system 10. As discussed more fully below, at least one of the conduits 50, 52 can be connected to a peripheral vessel, e.g., as a graft, using an anastomosis connection, and at least one of the conduits 50, 52 can be coupled with the same or another vessel via insertion of a cannula into the vasculature. Also, more than two conduits are used in some embodiments, as discussed below.

The inflow and outflow conduits 50, 52 may be formed from Dacron, Hemashield, Gortex, PVC, polyurethane, PTFE, ePTFE, nylon, or PEBAX materials, although other synthetic materials may be suitable. The inflow and outflow conduits 50, 52 may also comprise biologic materials or pseudobiological (hybrid) materials (e.g., biologic tissue supported on a synthetic scaffold). The inflow and outflow conduits 50, 52 are preferably configured to minimize kinks so blood flow is not meaningfully interrupted by normal movements of the patient or compressed easily from external forces. In some cases, the inflow and/or outflow conduits 50, 52 may come commercially already attached to the pump 32. Where it is desired to implant the pump 32 and the conduits 50, 52, it is preferable that the inner diameter of the conduits 50, 52 be less than 25 mm, although diameters slightly larger may be effective.

In one preferred application, the heart assist system 10 is applied in an arterial-arterial fashion; for example, as a femoral-axillary connection, as is shown in FIG. 1. It should be appreciated by one of ordinary skill in the art that an axillary-femoral connection would also be effective using the embodiments described herein. Indeed, it should be recognized by one of ordinary skill in the art that the present invention might be applied to any of the peripheral blood vessels in the patient. Another application of the heart assist system 10 couples the conduits 50, 52 with the same non-primary vessel in a manner similar to the application shown in FIG. 8 and discussed below.

FIG. 1 shows that the inflow conduit 50 has a first end 56 that connects with the inlet 34 of the pump 32 and a second end 58 that is coupled with a first non-primary blood vessel (e.g., the left femoral artery 26) by way of an inflow cannula 60. The inflow cannula 60 has a first end 62 and a second end 64. The first end 62 is sealably connected to the second end 58 of the inflow conduit 50. The second end 64 is inserted into the blood vessel (e.g., the left femoral artery 26). Although shown as discrete structures in FIG. 1, one skilled in the art would recognize that the inflow conduit 50 and the cannula 60 may be unitary in construction. The cannula 60 may take any suitable form, e.g., including one or more of the features of the cannulae discussed below in connection with FIGS. 17-26.

Where the conduit 50 is at least partially extracorporeal, the inflow cannula 60 also may be inserted through a surgical opening (e.g., as shown in FIG. 6 and described in connection therewith) or percutaneously, with or without an introducer sheath (not shown). In other applications, the inflow cannula 60 could be inserted into the right femoral artery or any other peripheral artery.

FIG. 1 shows that the outflow conduit 52 has a first end 66 that connects to the outlet 36 of the pump 32 and a second end 68 that connects with a second peripheral blood vessel, preferably the left subclavian artery 24 of the patient 12, although the right axillary artery, or any other peripheral artery, would be acceptable. In one application, the connection between the outflow conduit 52 and the second blood vessel is via an end-to-side anastomosis, although a side-to-side anastomosis connection might be used mid-stream of the conduit where the outflow conduit were connected at its second end to yet another blood vessel or at another location on the same blood vessel (neither shown). Preferably, the outflow conduit 52 is attached to the second blood vessel at an angle that results in the predominant flow of blood out of the pump 32 proximally toward the aorta 16 and the heart 14, such as is shown in FIG. 1, while still maintaining sufficient flow distally toward the hand to prevent limb ischemia.

In another embodiment, the inflow conduit 50 is connected to the first blood vessel via an end-to-side anastomosis, rather than via the inflow cannula 60. The inflow conduit 50 could also be coupled with the first blood vessel via a side-to-side anastomosis connection mid-stream of the conduit where the inflow conduit were connected at its second end to an additional blood vessel or at another location on the same blood vessel (neither shown). Further details of these arrangements and other related applications are described in U.S. application Ser. No. 10/289,467, filed Nov. 6, 2002, the entire contents of which is hereby incorporated by reference in its entirety and made a part of this specification.

In another embodiment, the outflow conduit 52 also is coupled with the second blood vessel via a cannula, as shown in FIG. 6. This connection may be achieved in a manner similar to that shown in FIG. 1 in connection with the first blood vessel.

It is preferred that application of the heart assist system 10 to the peripheral or non-primary blood vessels be accomplished subcutaneously; e.g., at a shallow depth just below the skin or first muscle layer so as to avoid major invasive surgery. It is also preferred that the heart assist system 10 be applied extrathoracically to avoid the need to invade the patient's chest cavity. Where desired, the entire heart assist system 10 may be implanted within the patient 12, either extravascularly, e.g., as in FIG. 1, or at least partially intravascularly, e.g., as in FIGS. 14-16.

In the case of an extravascular application, the pump 32 may be implanted, for example, into the groin area, with the inflow conduit 50 fluidly connected subcutaneously to, for example, the femoral artery 26 proximate the pump 32. The outflow conduit would be tunneled subcutaneously through to, for example, the left subclavian artery 24. In an alternative arrangement, the pump 32 and associated drive and controller could be temporarily fastened to the exterior skin of the patient, with the inflow and outflow conduits 50, 52 connected percutaneously. In either case, the patient may be ambulatory without restriction of tethered lines.

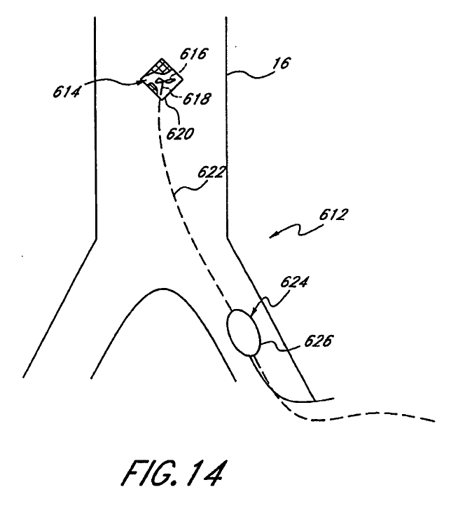

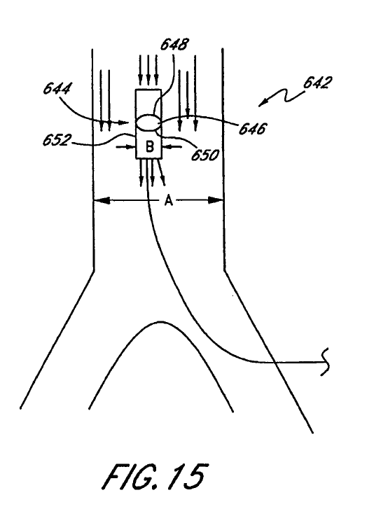

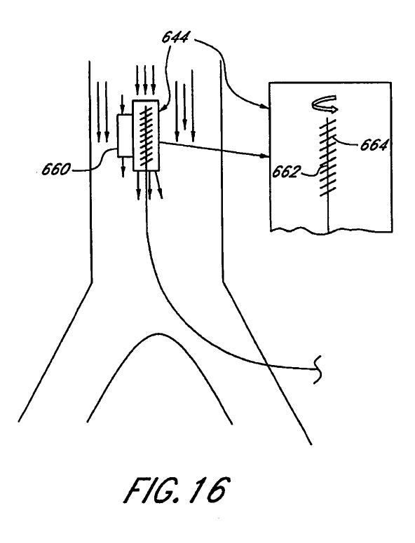

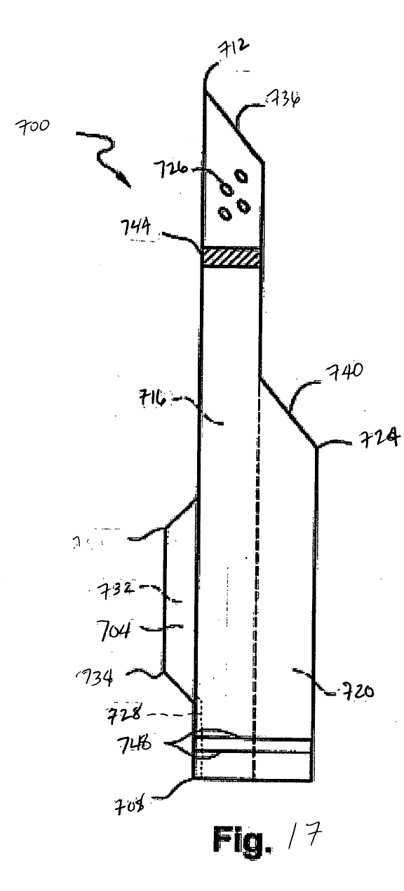

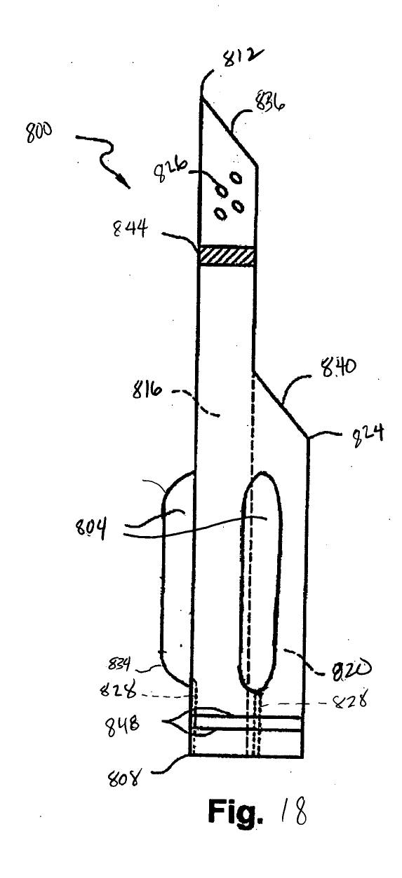

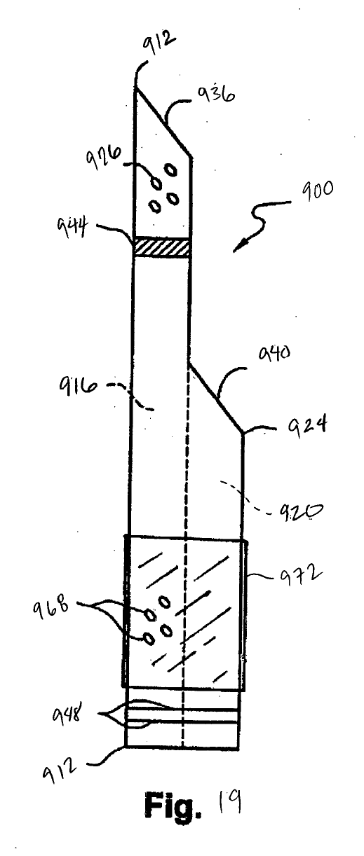

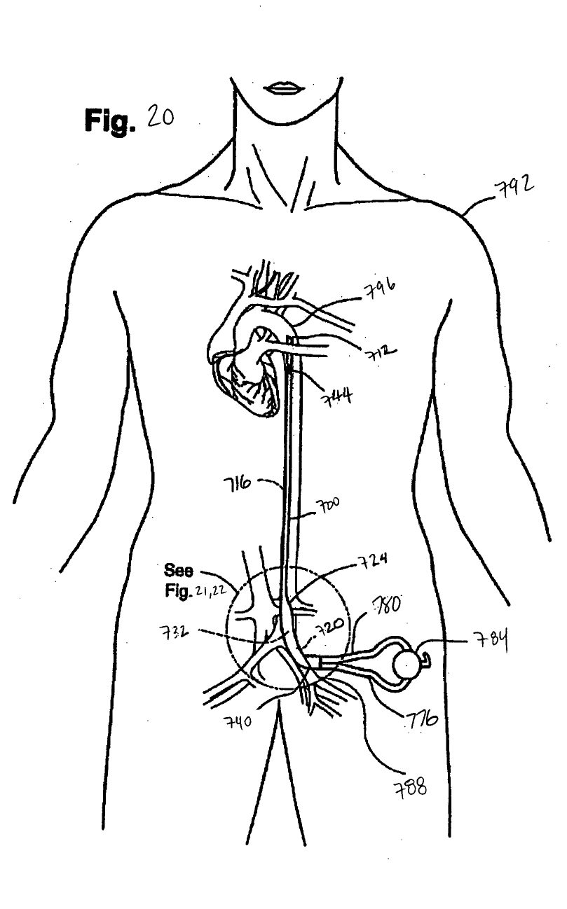

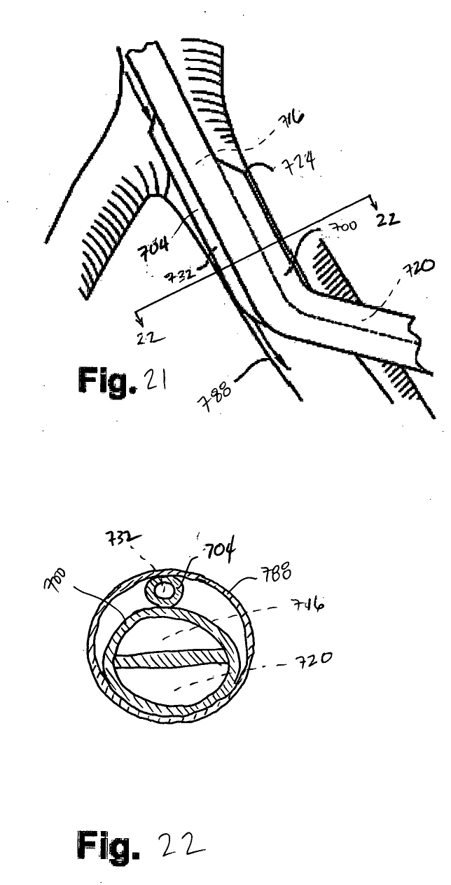

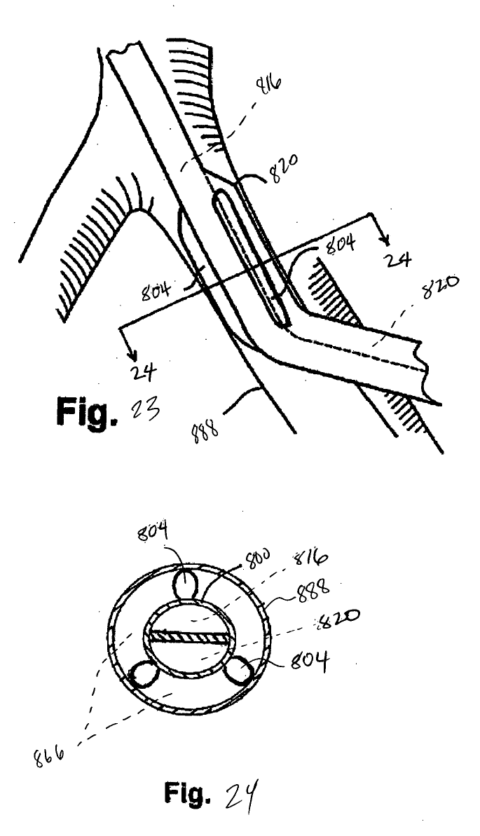

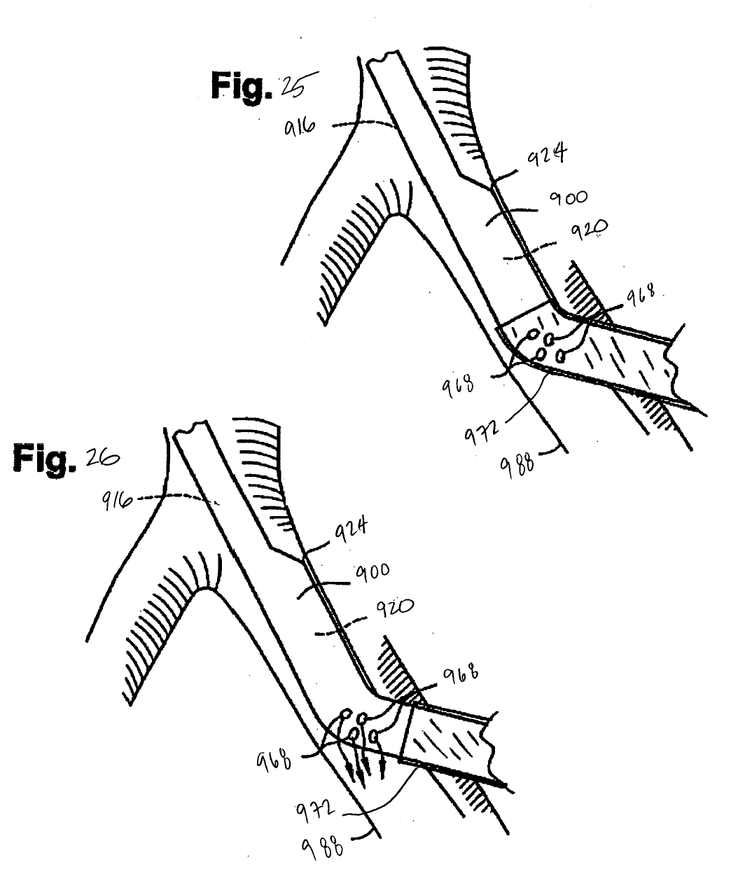

While the heart assist system 10 and other heart assist systems described herein may be applied to create an arterial-arterial flow path, given the nature of the heart assist systems, i.e., supplementation of circulation to meet organ demand, a venous-arterial flow path may also be used. For example, with reference to FIG. 2, one application of the heart assist system 10 couples the inflow conduit 50 with a non-primary vein of the patient 12, such as the left femoral vein 30. In this arrangement, the outflow conduit 50 may be fluidly coupled with one of the peripheral arteries, such as the left subclavian artery 24. Arterial-venous arrangements are contemplated as well. In those venous-arterial cases where the inflow is connected to a vein and the outflow is connected to an artery, the pump 32 should be sized to permit flow sufficiently small so that oxygen-deficient blood does not rise to unacceptable levels in the arteries. It should be appreciated that the connections to the non-primary veins could be by one or more approach described above for connecting to a non-primary artery. It should also be appreciated that the present invention could be applied as a venous-venous flow path, wherein the inflow and outflow are connected to separate peripheral veins. In addition, an alternative embodiment comprises two discrete pumps and conduit arrangements, one being applied as a venous-venous flow path, and the other as an arterial-arterial flow path.

When venous blood is mixed with arterial blood either at the inlet of the pump or the outlet of the pump the ratio of venous blood to arterial blood should be controlled to maintain an arterial saturation of a minimum of 80% at the pump inlet or outlet. Arterial saturation can be measured and/or monitored by pulse oximetry, laser doppler, colorimetry or other methods used to monitor blood oxygen saturation. The venous blood flow into the system can then be controlled by regulating the amount of blood allowed to pass through the conduit from the venous-side connection.

FIG. 3 shows another embodiment of a heart assist system 110 applied to the patient 12. For example, the heart assist system 110 includes a pump 132 in fluid communication with a plurality of inflow conduits 150A, 150B and a plurality of outflow conduits 152A, 152B. Each pair of conduits converges at a generally Y-shaped convergence 196 that converges the flow at the inflow end and diverges the flow at the outflow end. Each conduit may be connected to a separate peripheral blood vessel, although it is possible to have two connections to the same blood vessel at remote locations. In one arrangement, all four conduits are connected to peripheral arteries. In another arrangement, one or more of the conduits could be connected to veins. In the arrangement of FIG. 3, the inflow conduit 150A is connected to the left femoral artery 26 while the inflow conduit 150B is connected to the left femoral vein 30. The outflow conduit 152A is connected to the left subclavian artery 24 while the outflow conduit 152B is connected to the left carotid artery 22. Preferably at least one of the conduits 150A, 150B, 152A, and 152B is coupled with a corresponding vessel via a cannula. In the illustrated embodiment, the inflow conduit 150B is coupled with the left femoral vein 30 via a cannula 160. The cannula 160 is coupled in a manner similar to that shown in FIG. 2 and described in connection with the cannula 60. The cannula 160 preferably takes any suitable form, e.g., including one or more of the features of the cannulae discussed below in connection with FIGS. 17-26.

The connections of any or all of the conduits of the system 110 to the blood vessels may be via an anastomosis connection or via a connector, as described below in connection with FIG. 4. In addition, the embodiment of FIG. 3 may be applied to any combination of peripheral blood vessels that would best suit the patient's condition. For example, it may be desired to have one inflow conduit and two outflow conduits or vice versa. It should be noted that more than two conduits may be used on the inflow or outflow side, where the number of inflow conduits is not necessarily equal to the number of outflow conduits.

It is contemplated that, where an anastomosis connection is not desired, a connector may be used to connect at least one of the inflow conduit and the outflow conduit to a peripheral blood vessel. With reference to FIG. 4, an embodiment of a heart assist system 210 is shown, wherein an outflow conduit 252 is connected to a non-primary blood vessel, e.g., the left subclavian artery 24, via a connector 268 that comprises a three-opening fitting. In one embodiment, the connector 268 comprises an intra-vascular, generally T-shaped fitting 270 having a proximal end 272 (with respect to the flow of blood in the left axillary artery and therethrough), a distal end 274, and an angled divergence 276 permitting connection to the outflow conduit 252 and the left subclavian artery 24. The proximal and distal ends 274, 276 of the fittings 272 permit connection to the blood vessel into which the fitting is positioned, e.g., the left subclavian artery 24. The angle of divergence 276 of the fittings 272 may be 90 degrees or less in either direction from the axis of flow through the blood vessel, as optimally selected to generate the needed flow distally toward the hand to prevent limb ischemia, and to insure sufficient flow and pressure toward the aorta to provide the circulatory assistance and workload reduction needed while minimizing or avoiding endothelial damage to the blood vessel. In another embodiment, the connector 268 is a sleeve (not shown) that surrounds and attaches to the outside of the non-primary blood vessel where, within the interior of the sleeve, a port to the blood vessel is provided to permit blood flow from the outflow conduit 252 when the conduit 252 is connected to the connector 268.

Other types of connectors having other configurations are contemplated that may avoid the need for an anastomosis connection or that permit connection of the conduit(s) to the blood vessel(s). For example, it is contemplated that an L-shaped connector be used if it is desired to withdraw blood more predominantly from one direction of a peripheral vessel or to direct blood more predominantly into a peripheral vessel. Referring to FIG. 5, the inflow conduit 250 is fluidly connected to a peripheral vessel, for example, the left femoral artery 26, using an L-shaped connector 278. Of course the system 210 could be configured so that the outflow conduit 252 is coupled to a non-primary vessel via the L-shaped connector 278 and the inflow conduit 250 is coupled via a cannula, as shown in FIG. 3. The L-shaped connector 278 has an inlet port 280 at a proximal end and an outlet port 282 through which blood flows into the inflow conduit 250. The L-shaped connector 278 also has an arrangement of holes 284 within a wall positioned at a distal end opposite the inlet port 280 so that some of the flow drawn into the L-shaped connector 278 is diverted through the holes 284, particularly downstream of the L-shaped connector 278, as in this application. A single hole 284 in the wall could also be effective, depending upon size and placement. The L-shaped connector 278 may be a deformable L-shaped catheter percutaneously applied to the blood vessel or, in an alternative embodiment, be connected directly to the walls of the blood vessel for more long term application. By directing some blood flow downstream of the L-shaped connector 278 during withdrawal of blood from the vessel, ischemic damage downstream from the connector may be avoided. Such ischemic damage might otherwise occur if the majority of the blood flowing into the L-shaped connector 278 were diverted from the blood vessel into the inflow conduit 252. It is also contemplated that a connection to the blood vessels might be made via a cannula, wherein the cannula is implanted, along with the inflow and outflow conduits.

One advantage of discrete connectors manifests in their application to patients with chronic CHF. A connector eliminates a need for an anastomosis connection between the conduits 250, 252 and the peripheral blood vessels where it is desired to remove and/or replace the system more than one time. The connectors could be applied to the first and second blood vessels semi-permanently, with an end cap applied to the divergence for later quick-connection of the present invention system to the patient. In this regard, a patient might experience the benefit of the heart assist systems described herein periodically, without having to reconnect and redisconnect the conduits 250, 252 from the blood vessels via an anastomosis procedure each time. Each time it is desired to implement any of the embodiments of the heart assist system, the end caps would be removed and a conduit attached to the connector(s) quickly.

In the preferred embodiment of the connector 268, the divergence 276 is oriented at an acute angle significantly less than 90 degrees from the axis of the T-shaped fitting 270, as shown in FIG. 4, so that a majority of the blood flowing through the outflow conduit 252 into the blood vessel (e.g., left subclavian artery 24) flows in a direction proximally toward the heart 14, rather than in the distal direction. In an alternative embodiment, the proximal end 272 of the T-shaped fitting 270 may have a diameter larger than the diameter of the distal end 274, without need of having an angled divergence, to achieve the same result.

With or without a connector, with blood flow directed proximally toward the aorta 16, the result may be concurrent flow down the descending aorta, which will result in the reduction of afterload, impedence, and/or reducing left ventricular end diastolic pressure and volume (preload). Thus, the heart assist systems described herein may be applied so to reduce the afterload on the patient's heart, permitting at least partial if not complete CHF recovery, while supplementing blood circulation. Concurrent flow depends upon the phase of operation of the pulsatile pump and the choice of second blood vessel to which the outflow conduit is connected.

A partial external application of the heart assist systems is contemplated where a patient with heart failure is suffering an acute decompensation episode; i.e., is not expected to last long, or in the earlier stages of heart failure (where the patient is in New York Heart Association Classification (NYHAC) functional classes II or III). With reference to FIGS. 6 and 7, another embodiment of a heart assist system 310 is applied percutaneously to a patient 312 to connect two non-primary blood vessels wherein a pump 332 and its associated driving means and controls are employed extracorporeally. The pump 332 has an inflow conduit 350 and an outflow conduit 352 associated therewith for connection to two non-primary blood vessels. The inflow conduit 350 has a first end 356 and a second end 358 wherein the second end 358 is connected to a first non-primary blood vessel (e.g., femoral artery 26) by way of an inflow cannula 380. The inflow cannula 380 has a first end 382 sealably connected to the second end 358 of the inflow conduit 350. The inflow cannula 380 also has a second end 384 that is inserted through a surgical opening 386 or an introducer sheath (not shown) and into the blood vessel (e.g., the left femoral artery 26).

Similarly, the outflow conduit 352 has a first end 362 and a second end 364 wherein the second end 364 is connected to a second non-primary blood vessel (e.g., the left subclavian artery 24, as shown in FIG. 6, or the right femoral artery 28, as shown in FIG. 7) by way of an outflow cannula 388. Like the inflow cannula 380, the outflow cannula 388 has a first end 390 sealably connected to the second end 364 of the outflow conduit 352. The outflow cannula 388 also has a second end 392 that is inserted through surgical opening 394 or an introducer sheath (not shown) and into the second blood vessel (e.g., the left subclavian artery 24 or the right femoral artery 28). The cannulae 380 and 388 preferably take any suitable form. The cannulae 380, 388 may take any suitable form, e.g., including one or more of the features of the cannulae discussed below in connection with FIGS. 17-26.

As shown in FIG. 7, the second end 392 of the outflow cannula 388 may extend well into the aorta 16 of the patient 12, for example, proximal to the left subclavian artery. If desired, it may also terminate within the left subclavian artery or the left axillary artery, or in other blood vessels, such as the mesenteric or renal arteries (not shown), where in either case, the outflow cannula 388 has passed through at least a portion of a primary artery (in this case, the aorta 16). Also, if desired, blood drawn into the extracardiac system 310 described herein may originate from the descending aorta (or an artery branching therefrom) and be directed into a blood vessel that is neither the aorta nor pulmonary artery. By use of a percutaneous application, the heart assist system 310 may be applied temporarily without the need to implant any aspect thereof or to make anastomosis connections to the blood vessels.

An alternative variation of the embodiment of FIG. 6 may be used where it is desired to treat a patient periodically, but for short periods of time each occasion and without the use of special connectors. With this variation, it is contemplated that the second ends of the inflow and outflow conduits 350, 352 be more permanently connected to the associated blood vessels via, for example, an anastomosis connection, wherein a portion of each conduit proximate to the blood vessel connection is implanted percutaneously with a removable cap enclosing the externally-exposed first end (or an intervening end thereof) of the conduit external to the patient. When it is desired to provide a circulatory flow path to supplement blood flow, the removable cap on each exposed percutaneously-positioned conduit could be removed and the pump (or the pump with a length of inflow and/or outflow conduit attached thereto) inserted between the exposed percutaneous conduits. In this regard, a patient may experience the benefit of the present invention periodically, without having to reconnect and redisconnect the conduits from the blood vessels each time.

Specific methods of applying this alternative embodiment may further comprise coupling the inflow conduit 352 upstream of the outflow conduit 350 (as shown in FIG. 8), although the reverse arrangement is also contemplated. It is also contemplated that either the cannula 380 coupled with the inflow conduit 350 or the cannula 388 coupled with the outflow conduit 352 may extend through the non-primary blood vessel to a second blood vessel (e.g., through the left femoral artery 26 to the aorta 16 proximate the renal branch) so that blood may be directed from-the non-primary blood vessel to the second blood vessel or vice versa.

It is contemplated that a means for minimizing the loss of thermal energy in the patient's blood be provided where any of the heart assist systems described herein are applied extracorporeally. Such means for minimizing the loss of thermal energy may comprise, for example, a heated bath through which the inflow and outflow conduits pass or, alternatively, thermal elements secured to the exterior of the inflow and outflow conduits. Referring to FIG. 9, one embodiment comprises an insulating wrap 396 surrounding the outflow conduit 352 having one or more thermal elements passing therethrough. The elements may be powered, for example, by a battery (not shown). One advantage of thermal elements is that the patient may be ambulatory, if desired. Other means that are known by persons of ordinary skill in the art for ensuring that the temperature of the patient's blood remains at acceptable levels while travelling extracorporeally are also contemplated.

If desired, the present inventive system may further comprise a reservoir that is either contained within or in fluid communication with the inflow conduit. This reservoir is preferably made of materials that are nonthrombogenic. Referring to FIG. 9, a reservoir 398 is positioned fluidly in line with the inflow conduit 350. The reservoir 398 serves to sustain adequate blood in the system when the pump demand exceeds momentarily the volume of blood available in the peripheral blood vessel in which the inflow conduit resides until the pump output can be adjusted. The reservoir 398 reduces the risk of excessive drainage of blood from the peripheral blood vessel, which may occur when cardiac output falls farther than the already diminished baseline level of cardiac output, or when there is systemic vasodilation, as can occur, for example, with septic shock. It is contemplated that the reservoir 398 would be primed with an acceptable solution, such as saline, when the present system is first applied to the patient.

As explained above, one of the advantages of several embodiments of the heart assist system is that such systems permit the patient to be ambulatory. If desired, the systems may be designed portably so that it may be carried directly on the patient. Referring to FIG. 9, this may be accomplished through the use of a portable case 400 with a belt strap 402 to house the pump, power supply and/or the controller, along with certain portions of the inflow and/or outflow conduits, if necessary. It may also be accomplished with a shoulder strap or other techniques, such as a backpack or a fanny pack, that permit effective portability. As shown in FIG. 9, blood is drawn through the inflow conduit 350 into a pump contained within the portable case 400, where it is discharged into the outflow conduit 352 back into the patient.

B. Heart Assist Systems and Methods Employing Single-Site Application

As discussed above, heart assist systems can be applied to a patient through a single cannulation site. Such single-site systems can be configured with a pump located outside the vasculature of a patient, e.g., as extravascular pumping systems, inside the vasculature of the patient, e.g., as intravascular systems, or a hybrid thereof, e.g., partially inside and partially outside the vasculature of the patient.

1. Single-Site Application of Extravascular Pumping Systems

FIGS. 10 and 11 illustrate extracardiac heart assist systems that employ an extravascular pump and that can be applied through as a single-site system. FIG. 10 shows a system 410 that is applied to a patient 12 through a single cannulation site 414 while inflow and outflow conduits fluidly communicate with non-primary vessels. The heart assist system 410 is applied to the patient 12 percutaneously through a single site to couple two blood vessels with a pump 432. The pump 432 can have any of the features described in connection the pump 32. The pump 432 has an inflow conduit 450 and an outflow conduit 452 associated therewith. The inflow conduit 450 has a first end 456 and a second end 458. The first end 456 of the inflow conduit 450 is connected to the inlet of the pump 432 and the second end 458 of the inflow conduit 450 is fluidly coupled with a first non-primary blood vessel (e.g., the femoral artery 26) by way of a multilumen cannula 460. Similarly, the outflow conduit 452 has a first end 462 and a second end 464. The first end 462 of the outflow conduit 452 is connected to the outlet of the pump 432 and the second end 464 of the outflow conduit 452 is fluidly coupled with a second blood vessel (e.g., the descending aorta 16) by way of the multilumen cannula 460.

In one embodiment, the multilumen cannula 460 includes a first lumen 466 and a second lumen 468. The first lumen 466 extends from a proximal end 470 of the multilumen cannula 460 to a first distal end 472. The second lumen 468 extends from the proximal end 470 to a second distal end 474. In the illustrated embodiment, the second end 458 of the inflow conduit 450 is connected to the first lumen 466 of the multilumen cannula 460 and the second end 464 of the outflow conduit 452 is connected to the second lumen 468 of the multilumen cannula 460.

Where there is a desire for the patient 12 to be ambulatory, the multilumen cannula 460 preferably is made of material sufficiently flexible and resilient to permit the patient 12 to be comfortably move about while the multilumen cannula 460 is indwelling in the patient's blood vessels without causing any vascular trauma.

The application shown in FIG. 10 and described above results in flow from the first distal end 472 to the second distal end 474. Of course, the flow direction may be reversed using the same arrangement, resulting in flow from the distal end 474 to the distal end 472. In some applications, the system 410 is applied in an arterial-arterial fashion. For example, as illustrated, the multilumen cannula 460 can be inserted into the left femoral artery 26 of the patient 12 and guided superiorly through the descending aorta to one of numerous locations. In one application, the multilumen cannula 460 can be advanced until the distal end 474 is located in the aortic arch 476 of the patient 12. The blood could discharge, for example, directly into the descending aorta proximate an arterial branch, such as the left subclavian artery or directly into the peripheral mesenteric artery (not shown).

The pump 432 draws blood from the patient's vascular system in the area near the distal end 472 and into the lumen 466. This blood is further drawn into the lumen of the conduit 450 and into the pump 432. The pump 432 then expels the blood into the lumen of the outflow conduit 452, which carries the blood into the lumen 468 of the multilumen cannula 460 and back into the patient's vascular system in the area near the distal end 474.

FIG. 11 shows another embodiment of a heart assist system 482 that is similar to the heart assist system 410, except as set forth below. The system 482 employs a multilumen cannula 484. In one application, the multilumen cannula 484 is inserted into the left femoral artery 26 and guided superiorly through the descending aorta to one of numerous locations. Preferably, the multilumen cannula 484 has an inflow port 486 that is positioned in one application within the left femoral artery 26 when the cannula 484 is fully inserted so that blood drawn from the left femoral artery 26 is directed through the inflow port 486 into a first lumen 488 in the cannula 484. The inflow port 486 can also be positioned in any other suitable location within the vasculature, described herein or apparent to one skilled in the art. This blood is then pumped through a second lumen 490 in the cannula 484 and out through an outflow port 492 at the distal end of the cannula 484. The outflow port 492 may be situated within, for example, a mesenteric artery 494 such that blood flow results from the left femoral artery 26 to the mesenteric artery 494. The blood could discharge, for example, directly into the descending aorta proximate an arterial branch, such as the renal arteries, the left subclavian artery, or directly into the peripheral mesenteric artery 494, as illustrated in FIG. 11. Where there is a desire for the patient to be ambulatory, the multilumen cannula 484 preferably is made of material sufficiently flexible and resilient to permit the patient 12 to comfortably move about while the cannula 484 is indwelling in the patient's blood vessels without causing any vascular trauma. Further details of various embodiments of the multilumen cannula 460 are described below in connection with FIGS. 17-26.

FIG. 12 shows another heart assist system 510 that takes further advantage of the supplemental blood perfusion and heart load reduction benefits while remaining minimally invasive in application. The heart assist system 510 is an extracardiac pumping system that includes a pump 532, an inflow. conduit 550 and an outflow conduit 552. In the illustrated embodiment, the inflow conduit 550 comprises a vascular graft. The vascular graft conduit 550 and the outflow conduit 552 are fluidly coupled to pump 532. The pump 532 is configured to pump blood through the patient at subcardiac volumetric rates, and has an average flow rate that, during normal operation thereof, is substantially below that of the patient's heart when healthy. In one variation, the pump 532 may be a rotary pump. Other pumps described herein, or any other suitable pump can also be used in the extracardiac pumping system 510. In one application, the pump 532 is configured so as to be implantable.

The vascular graft 550 has a first end 554 and a second end 556. The first end 554 is sized and configured to couple to a non-primary blood vessel 558 subcutaneously to permit application of the extracardiac pumping system 510 in a minimally-invasive procedure. In one application, the vascular graft conduit 550 is configured to couple to the blood vessel 558 via an anastomosis connection. The second end 556 of the vascular graft 550 is fluidly coupled to the pump 532 to conduct blood between the non-primary blood vessel 558 and the pump 532. In the embodiment shown, the second end 556 is directly connected to the pump 532, but, as discussed above in connection with other embodiments, intervening fluid conducting elements may be interposed between the second end 556 of the vascular graft 550 and the pump 532. Examples of arrangements of vascular graft conduits may be found in U.S. application Ser. No. 09/780,083, filed Feb. 9, 2001, entitled EXTRA-CORPOREAL VASCULAR CONDUIT, which is hereby incorporated by reference in its entirety and made a part of this specification.

FIG. 12 illustrates that the present inventive embodiment further comprises means for coupling the outflow conduit 552 to the vascular graft 550, which may comprise in one embodiment an insertion site 560. In the illustrated embodiment, the insertion site 560 is located between the first end 554 and the second end 556 of the vascular graft 550. The outflow conduit 552 preferably is coupled with a cannula 562. The cannula 562 may take any suitable form, e.g., incorporating one or more of the features of the cannulae discussed below in connection with FIGS. 17-26.

The insertion site 560 is configured to receive the cannula 562 therethrough in a sealable manner in the illustrated embodiment. In another embodiment, the insertion site 560 is configured to receive the outflow conduit 552 directly. The cannula 562 includes a first end 564 sized and configured to be inserted through the insertion site 560, through the cannula 550, and through the non-primary blood vessel 558. The conduit 552 has a second end 566 fluidly coupled to the pump 532 to conduct blood between the pump 532 and the blood vessel 558.

The extracardiac pumping system 510 can be applied to a patient, as shown in FIG. 12, so that the outflow conduit 552 provides fluid communication between the pump 532 and a location upstream or downstream of the point where the cannula 562 enters the non-primary blood vessel 558. In another application, the cannula 562 is directed through the blood vessel to a different blood vessel, upstream or downstream thereof. Although the vascular graft 550 is described above as an “inflow conduit” and the conduit 552 is described above as an “outflow conduit,” in another application of this embodiment, the blood flow through the pumping system 510 is reversed (i.e., the pump 532 pumps blood in the opposite direction), whereby the vascular graft 550 is an outflow conduit and the conduit 552 is an inflow conduit.

FIG. 13 shows a variation of the extracardiac pumping system shown in FIG. 12. In particular, a heart assist system 570 includes an inflow conduit 572 that comprises a first end 574, a second end 576, and means for connecting the outflow conduit 552 to the inflow conduit 572. In one embodiment, the inflow conduit 572 comprises a vascular graft. The extracardiac pumping system 570 is otherwise similar to the extracardiac pumping system 510. The means for connecting the conduit 552 to the inflow conduit 572 may comprise a branched portion 578. In one embodiment, the branched portion 578 is located between the first end 574 and the second end 576. The branched portion 578 is configured to sealably receive the distal end 564 of the outflow conduit 552. Where, as shown, the first end 564 of the outflow conduit 552 comprises the cannula 562, the branched portion 578 is configured to receive the cannula 562. The inflow conduit 572 of this arrangement comprises in part a multilumen cannula, where the internal lumen extends into the blood vessel 558. Other multilumen catheter arrangements are shown in U.S. application Ser. No. 10/078,283, incorporated by reference herein above.

2. Single-Site Application of Intravascular Pumping Systems

FIGS. 14-16 illustrate extracardiac heart assist systems that employ intravascular pumping systems. Such systems take further advantage of the supplemental blood perfusion and heart load reduction benefits discussed above while remaining minimally invasive in application. Specifically, it is contemplated to provide an extracardiac pumping system that comprises a pump that is sized and configured to be at least partially implanted intravascularly in any location desirable to achieve those benefits, while being insertable through a non-primary vessel.

FIG. 14 shows a heart assist system 612 that includes a pumping means 614 comprising preferably one or more rotatable impeller blades 616, although other types of pumping means 614 are contemplated, such as an archimedes screw, a worm pump, or other means by which blood may be directed axially along the pumping means from a point upstream of an inlet to the pumping means to a point downstream of an outlet from the pumping means. Where one or more impeller blades 616 are used, such as in a rotary pump, such impeller blades 616 may be supported helically or otherwise on a shaft 618 within a housing 620. The housing 620 may be open, as shown, in which the walls of the housing 620 are open to blood flow therethrough. The housing 620 may be entirely closed, if desired, except for an inlet and outlet (not shown) to permit blood flow therethrough in a more channel fashion. For example, the housing 620 could be coupled with or replaced by a cannula with a downstream blood flow enhancing portion, such as those illustrated in FIGS. 17-26. The heart assist system 612 serves to supplement the kinetic energy of the blood flow through the blood vessel in which the pump is positioned, e.g., the aorta 16.

The impeller blade(s) 616 of the pumping means 614 of this embodiment may be driven in one or a number of ways known to persons of ordinary skill in the art. In the embodiment shown in FIG. 14, the impeller blade(s) 616 are driven mechanically via a rotatable cable or drive wire 622 by driving means 624, the latter of which may be positioned corporeally (intra- or extra-vascularly) or extracorporeally. As shown, the driving means 624 may comprise a motor 626 to which energy is supplied directly via an associated battery or an external power source, in a manner described in more detail herein. It is also contemplated that the impeller blade(s) 616 be driven electromagnetically through an internal or external electromagnetic drive. Preferably, a controller (not shown) is provided in association with this embodiment so that the pumping means 614 may be controlled to operate in a continuous and/or pulsatile fashion, as described herein.

Variations of the intravascular embodiment of FIG. 14 are shown in FIGS. 15 and 16. In the embodiment of FIG. 15, an intrasvascular extracardiac system 642 comprising a pumping means 644, which may be one of several means described herein. The pumping means 644 may be driven in any suitable manner, including means sized and configured to be implantable and, if desired, implantable intravascularly, e.g., as discussed above. For a blood vessel (e.g., descending aorta) having a diameter “A”, the pumping means 644 preferably has a meaningfully smaller diameter “B”. The pumping means 644 may comprise a pump 646 having an inlet 648 and an outlet 650. The pumping means 644 also comprises a pump driven mechanically by a suitable drive arrangement in one embodiment. Although the vertical arrows in FIG. 15 illustrate that the pumping means 644 pumps blood in the same direction as the flow of blood in the vessel, the pumping means 644 could be reversed to pump blood in a direction generally opposite of the flow in the vessel.

In one embodiment, the pumping means 644 also includes a conduit 652 in which the pump 646 is housed. The conduit 652 may be relatively short, as shown, or may extend well within the designated blood vessel or even into an adjoining or remote blood vessel at either the inlet end, the outlet end, or both. The intravascular extracardiac system 642 may further comprise an additional parallel-flow conduit, as discussed below in connection with the system of FIG. 16.

The intrasvascular extracardiac system 642 may further comprise inflow and/or outflow conduits or cannulae (not shown) fluidly connected to the pumping means 644, e.g., to the inlet and outlet of pump 646. Any suitable conduit or cannula can be employed. For example, a cannula having a downstream blood flow enhancing portion, such as the any of the cannulae of FIGS. 17-26, could be coupled with an intrasvascular extracardiac system.

In another embodiment, an intrasvascular pumping means 644 may be positioned within one lumen of a multilumen catheter so that, for example, where the catheter is applied at the left femoral artery, a first lumen may extend into the aorta proximate the left subclavian and the pumping means may reside at any point within the first lumen, and the second lumen may extend much shorter just into the left femoral or left iliac. Such a system is described in greater detail in U.S. application Ser. No. 10/078,283, incorporated by reference herein above.

FIG. 16 shows a variation of the heart assist system of FIG. 15. In particular the intravascular system may further comprise an additional conduit 660 positioned preferably proximate the pumping means 644 to provide a defined flow path for blood flow axially parallel to the blood flowing through the pumping means 644. In the case of the pumping means 644 of FIG. 16, the means comprises a rotatable cable 662 having blood directing means 664 supported therein for directing blood axially along the cable. Other types of pumping means are also contemplated, if desired, for use with the additional conduit 660.

The intravascular extracardiac system described herein may be inserted into a patient's vasculature in any means known by one of ordinary skill or obvious variant thereof. In one method of use, such a system is temporarily housed within a catheter that is inserted percutaneously, or by surgical cutdown, into a non-primary blood vessel and advanced through to a desired location. The catheter preferably is then withdrawn away from the system so as not to interfere with operation of the system, but still permit the withdrawal of the system from the patient when desired. Further details of intravascular pumping systems may be found in U.S. patent application Ser. No. 10/686,040, filed Oct. 15, 2003, which is hereby incorporated by reference herein in its entirety.

C. Potential Enhancement of Systemic Arterial Blood Mixing

One of the advantages of the present invention is its potential to enhance mixing of systemic arterial blood, particularly in the aorta. Such enhanced mixing ensures the delivery of blood with higher oxygen-carrying capacity to organs supplied by arterial side branches off of the aorta. A method of enhancing mixing utilizing the present invention preferably includes taking steps to assess certain parameters of the patient and then to determine the minimum output of the pump that, when combined with the heart output, ensures turbulent flow in the aorta, thereby enhancing blood mixing.

Blood flow in the aortic arch during normal cardiac output may be characterized as turbulent in the end systolic phase. It is known that turbulence in a flow of fluid through pipes and vessels enhances the uniform distribution of particles within the fluid. It is believed that turbulence in the descending aorta enhances the homogeneity of blood cell distribution in the aorta. It is also known that laminar flow of viscous fluids leads to a higher concentration of particulate in the central portion of pipes and vessels through which the fluid flows. It is believed that, in low flow states such as that experienced during heart failure, there is reduced or inadequate mixing of blood cells leading to a lower concentration of nutrients at the branches of the aorta to peripheral organs and tissues. As a result, the blood flowing into branch arteries off of the aorta will likely have a lower hematocrit, especially that flowing into the renal arteries, the celiac trunk, the spinal arteries, and the superior and inferior mesenteric arteries. That is because these branches draw from the periphery of the aorta The net effect of this phenomenon is that the blood flowing into these branch arteries has a lower oxygen-carrying capacity, because oxygen-carrying capacity is directly proportional to both hematocrit and the fractional O₂ saturation of hemoglobin. Under those circumstances, it is very possible that these organs will experience ischemia-related pathology.

The phenomenon of blood streaming in the aorta, and the resultant inadequate mixing of blood resulting in central lumenal concentration of blood cells, is believed to occur when the Reynolds number (N_{R}) for the blood flow in the aorta is below 2300. To help ensure that adequate mixing of blood will occur in the aorta to prevent blood cells from concentrating in the center of the lumen, a method of applying the present invention to a patient may also include steps to adjust the output of the pump to attain turbulent flow within the descending aorta upstream of the organ branches; i.e., flow exhibiting a peak Reynolds number of at least 2300 within a complete cycle of systole and diastole. Because flow through a patient is pulsatile in nature, and not continuous, consideration must be given to how frequently the blood flow through the aorta has reached a certain desired velocity and, thus, a desired Reynolds number. The method contemplated herein, therefore, should also include the step of calculating the average Womersley number (N_{W}), which is a function of the frequency of the patient's heart beat. It is desired that a peak Reynolds number of at least 2300 is attained when the corresponding Womersley number for the same blood flow is approximately 6 or above.

More specifically, the method may comprise calculating the Reynolds number for the blood flow in the descending aorta by determining the blood vessel diameter and both the velocity and viscosity of the fluid flowing through the aorta. The Reynolds number may be calculated pursuant to the following equation:

$${N}_{R} = \frac{V \cdot d}{ν}$$

where: V=the velocity of the fluid; d=the diameter of the vessel; and υ=the viscosity of the fluid. The velocity of the blood flowing through the aorta is a function of the cross-sectional area of the aorta and the volume of flow therethrough, the latter of which is contributed both by the patient's own cardiac output and by the output of the pump of the present invention. Velocity may be calculated by the following equation:

$$V = \frac{Q}{π {r}^{2}}$$

where Q=the volume of blood flowing through the blood vessel per unit time, e. g., the aorta, and r=radius of the aorta. If the relationship between the pump output and the velocity is already known or independently determinable, the volume of blood flow Q may consist only of the patient's cardiac output, with the knowledge that that output will be supplemented by the subcardiac pump that is part of the present invention. If desired, however, the present system can be implemented and applied to the patient first, before calculating Q, which would consist of the combination of cardiac output and the pump output.

The Womersley number may be calculated as follows:
N_{W}=r\sqrt{2πω/}_{υ}

where r is the radius of the vessel being assessed, ω is the frequency of the patient's heartbeat, and υ=the viscosity of the fluid. For a peak Reynolds number of at least 2300, a Womersley number of at least 6 is preferred, although a value as low as 5 would be acceptable.

By determining (i) the viscosity of the patient's blood, which is normally about 3.0 mm²/sec sec (kinematic viscosity), (ii) the cardiac output of the patient, which of course varies depending upon the level of CHF and activity, and (iii) the diameter of the patient's descending aorta, which varies from patient to patient but is about 21 mm for an average adult, one can determine the flow rate Q that would result in a velocity through the aorta necessary to attain a Reynolds number of at least 2300 at its peak during the patient's heart cycle. Based upon that determination of Q, one may adjust the output of the pump of the present invention to attain the desired turbulent flow characteristic through the aorta, enhancing mixing of the blood therethrough.

One may use ultrasound (e.g., echocardiography or abdominal ultrasound) to measure the diameter of the aorta, which is relatively uniform in diameter from its root to the abdominal portion of the descending aorta. Furthermore, one may measure cardiac output using a thermodilution catheter or other techniques known to those of skill in the art. Finally, one may measure viscosity of the patient's blood by using known methods; for example, using a capillary viscosimeter. It is expected that in many cases, the application of this embodiment of the present method will provide a basis to more finely tune the system to more optimally operate the system to the patient's benefit. Other methods contemplated by the present invention may include steps to assess other patient parameters that enable a person of ordinary skill in the art to optimize the present system to ensure adequate mixing within the vascular system of the patient.

Alternative inventive methods that provide the benefits discussed herein include the steps of, prior to applying a shape change therapy, applying a blood supplementation system (such as one of the many examples described herein) to a patient, whereby the methods are designed to improve the ability to reduce the size and/or wall stress of the left ventricle, or both ventricles, thus reducing ventricular loading. Specifically, one example of such a method comprises the steps of providing a pump configured to pump blood at subcardiac rates, providing inflow and outflow conduits configured to fluidly communicate with-non-primary blood vessels, fluidly coupling the inflow conduit to a non-primary blood vessel, fluidly coupling the outflow conduit to the same or different (primary or non-primary) blood vessel and operating the subcardiac pump in a manner, as described herein, to reduce the load on the heart, wherein the fluidly coupling steps may comprise anastomosis, percutaneous cannulazation, positioning the distal end of one or both conduits within the desired terminal blood vessel or any combination thereof. The method further comprises, after sufficient reduction in ventricular loading, applying a shape change therapy in the form of, for example, a cardiac reshaping device, such as those referred to herein, or others serving the same or similar function, for the purpose of further reducing the size of and/or wall stress on one or more ventricles and, thus, the heart, and/or for the purpose of maintaining the patient's heart at a size sufficient to enhance recovery of the patient's heart.

### II. Cannulae and Cannula System for Use in Heart Assit Systems

With reference to FIGS. 17-26, various embodiments of perfusion cannula systems comprise a cannula body and a means for enhancing blood flow past the cannula body when the cannula body resides within the patient. The enhancing means preferably is capable of selectively enhancing blood flow around the cannula body within the vasculature of the patient. For example, as shown in FIGS. 17 and 18, and discussed further below, in some embodiments, the enhancing means comprises at least one balloon. In other embodiments, as shown in FIG. 19, and discussed further below, the enhancing means comprises at least one aperture that can be selectively covered and uncovered by a sleeve.

With reference to FIG. 17, one embodiment of a perfusion cannula system includes a cannula 700 that is configured to direct blood through the vasculature of a patient. The cannula system also includes a balloon 704 that is coupled with the cannula 700. The balloon 704 preferably is located on the exterior of the cannula 700. In one embodiment, the cannula 700 and the balloon 704 are physically distinct, i.e., formed in separate processes and later coupled, and together form a catheter system. In other embodiments, the cannula 700 and the balloon 704 are formed together and the balloon 704 is considered to be a part of the cannula 700. As discussed in greater detail below, the balloon 704 may be deployed to provide space between a vessel wall and the cannula 700 when the cannula 700 resides within the patient. The balloon 704 may thereby enable or enhance passive perfusion of blood past the cannula 700. The term “passive perfusion” is used in its ordinary sense and is a broad term that includes providing a path for blood flow under prevailing blood pressure within the vessel and that is not otherwise externally assisted.

The cannula 700 comprises a proximal end 708, a distal end 712, and at least one lumen that extends therebetween. With reference to FIG. 17, the cannula 700 defines a first lumen 716 that extends between the proximal end 708 and the distal end 712 and also defines a second lumen 720 that extends between the proximal end 708 and a distal end 724. The lumens 716, 720 may provide for inflow and outflow of blood in connection with a heart assist system, such as those discussed above in connection with FIGS. 10-16. Although shown as a multilumen cannula, the cannula 700 could also be configured as a single lumen cannula, which could be employed in multi-site applications, such as those shown in FIGS. 1-9.

One or more apertures 726 may be formed in the cannula 700 proximate the distal end 712, although such apertures may also be formed proximate the distal end 724. The apertures 726 may be positioned close together or spaced circumferentially around the portion of the cannula 700 defining the lumen 716. The apertures 726 decrease the pressure drop across the distal end 712, thereby minimizing damage to vessel walls from jetting effects. Where one ore more apertures are formed proximate the distal end 724, the apertures decrease the pressure differential across the distal end 724, thereby minimizing the tendency of the vessel wall to be sucked into the distal end 724. Further tip arrangements that may be advantageously employed that provide desired outflow characteristics are described in more detail in U.S. patent application Ser. No. 10/706,346, filed Nov. 12, 2003, which is hereby expressly incorporated by reference herein in its entirety.

The lumens 716, 720 of the cannula 700 may be arranged in any of a number of different ways. For example, the two lumens may be joined in a side-by-side manner, forming a “figure-8” when viewed from the proximal end 708. In another embodiment, the cannula 700 may contain within it two or more side-by-side lumens. A cylindrical cannula body could be formed with a wall extending across the cylinder at a diameter to form two lumens. A cylindrical cannula body with concentrically positioned lumens is also contemplated.

The cannula system also includes an auxiliary lumen 728 that is in fluid communication with the balloon 704. The auxiliary lumen 728 may be defined in the body of the cannula 700. The lumen 728 preferably extends from the proximal end 708 of the cannula 700 to the balloon 704. The lumen 728 is referred to herein as an “auxiliary lumen” because it is generally substantially smaller than the lumens 716, 720 and because it enables a function that is not primary to the operation of the cannula 700. The lumen 728 is one means for deploying the balloon 704 within the vasculature and in one embodiment is an inflation lumen for the balloon 704. Preferably, the lumen 728 may be selectively fluidly coupled with a source of any suitable inflation media. The inflation media may be another means for deploying the balloon 704. The inflation media may include a suitable gas or liquid, such as saline. The inflation media may be delivered by way of a syringe (not shown), which is another means for deploying the balloon 704.

The balloon 704 is formed of an inflatable material that can be actuated from a deflated state to an inflated state. When in the deflated state, the balloon 704 preferably substantially conforms to at least a portion of the outside surface of the cannula 700. The balloon 704 is also one form of a collapsible element that can be selectively collapsed to ease insertion of the cannula system 700 into the vasculature. After being inserted into the patient, as described in more detail below, the balloon 704 may be inflated to the inflated state shown in FIG. 17. Thus, the balloon 704 is one form of an expandable element, e.g., one that may be selectively expanded to provide the function of passive perfusion, as discussed herein. Other forms of collapsible and expandable elements are also possible, such as those that employ a mechanically actuatable element and those that automatically collapse or expand, such as self-expanding elements.

In one embodiment, the balloon 704 has a tubular configuration when in the inflated state. The tubular configuration of the balloon 704 provides an inside surface that defines a perfusion lumen 732. The perfusion lumen 732 is a generally longitudinally extending lumen, e.g., one that is generally parallel to the lumens 716, 720. As shown in FIG. 22 and discussed in more detail below, the perfusion lumen 732 has a generally circular cross-section in one embodiment and is large enough to permit a substantial amount of blood to flow therethrough. The flow through the perfusion lumen 732 is directed beyond a proximal end 734 of the balloon 704 and beyond the insertion site of the cannula 700 into the vasculature downstream to tissue that might otherwise be deprived of oxygenated blood.

Additional features that may be incorporated into the cannula 700 include a tapered tip 736 at the first distal end 712 and/or a tapered tip 740 at the second distal end 724. The tapered tips 736, 740 may facilitate insertion and threading of the cannula 700 into the patient. The cannula 700 may also be provided with a radiopaque marker 744, which may be positioned proximate the distal end 712. The cannula 700 could further comprise markings 748 near the proximal end 708 and a known distance from one or more of the distal ends 712, 724. The markings 748, as well as the radiopaque marker 744, can be used to accurately position the cannula 700 when inserted within the patient.

With reference to FIG. 18, in another embodiment a cannula 800 comprises one or more inflatable members or balloons 804 extending between a proximal end 808 and a distal end 812. In the embodiment illustrated in FIG. 18, a plurality of balloons 804 are provided. The balloons 804 are positioned and sized such that when the cannula 800 resides in the patient (described below), the balloons 804 reside entirely within the patient's body. The balloons 804 are spaced radially about the cannula 800, e.g., equally spaced around the cannula 800. As described above, the balloons 804 may be connected to the cannula 800 in a variety of ways. The balloons 804 can be formed integrally with the cannula 800. The balloons 804 can also be formed separately and coupled to the cannula 800 in any suitable manner. One purpose of the balloons 804 is to provide passive perfusion, e.g., to selectively permit the passive flow of blood downstream to the cannula to enhance perfusion. The balloons 804 therefore comprise a means for creating space around the cannula 800 within the vasculature to permit blood flow past the cannula 800.

The balloons 804 are one form of an expandable element, e.g., one that may be selectively expanded to provide the function of passive perfusion, as discussed above. The balloon 804 is also one form of a collapsible element that is selectively collapsible to ease insertion of a cannula system into the vasculature. Other forms of collapsible and expandable elements are also possible, such as those that employ one or more mechanically actuatable elements and those that employ one or more elements that automatically collapse or expand, such as self-expanding elements.

The balloons 804 may be made of inflatable material, e.g., one capable of taking on an inflated and deflated state. In the deflated state, the balloons 804 would conform to at least a portion of the outside surface of the cannula 800. Once inserted within the patient, as described in more detail below, the balloons 804 would be inflated to the inflated state shown in FIG. 18. The inflatable balloons 804 can have any suitable configuration. Preferably, when the balloons 804 are deployed within a patient's body they contact the surface of the vessel wall. Here, the balloons 804 are used primarily to create a space between the cannula 800 and the vessel wall to permit the passive flow of blood downstream of the cannula site to enhance perfusion, e.g., to provide passive perfusion. Blood preferably flows through spaces formed alongside the inflated balloons 804 between the cannula 800 and a vessel wall. As described previously, the balloons 804 can be inflated by filling the balloons 804 with gas or liquid through auxiliary lumens 828 defined in the body of the cannula 800, or in any other suitable manner.

With reference to FIG. 19, in another embodiment, a cannula system 900 comprises a cannula 902 having an aperture 968 formed in the body thereof and a sleeve 972. In some embodiments a plurality of apertures 968 may be provided. The apertures 968 can be positioned on the cannula system 900 near the proximal end 912. The apertures 968 preferably are formed on the body of the cannula 902 and provide fluid communication between one of the lumens 916, 920 and the blood vessel in which the cannula 902 resides.

In one embodiment the sleeve 972 is carried by the cannula 902 and is configured to be moveable relative to the apertures 968 to selectively cover and uncover the apertures 968 as desired. The sleeve 972 can be carried on either the outside or the inside of the cannula 902. For example, when the apertures 968 are formed on the body of the cannula 902 to provide fluid communication between the lumen 916 and the blood vessel, the sleeve 972 could be carried within the lumen 916. The sleeve 972 could be carried within the lumen 920 in a similar fashion to selectively cover and uncover apertures formed in the body of the cannula 902 to provide fluid communication between the lumen 920 and the blood vessel. In the illustrated embodiment, the sleeve 972 is on the outside of the body of the cannula 902. The sleeve 972 can be configured to move radially with respect to the cannula 902. The sleeve 972 can also be configured to move longitudinally, e.g., distally or proximally, with respect to the cannula 902.

The apertures 968 can be selectively uncovered while the cannula system 900 resides within a patient's body. Here, the sleeve 972 and apertures 968 are used primarily to selectively provide active perfusion of blood downstream of the location of the cannula 902 within the blood vessel. As used herein “active perfusion” is used in its ordinary sense and is a broad term that includes providing additional flow of blood under external blood pressure, e.g., the blood pressure generated by a pump forcing blood into the lumen 916, into the vessel to increase downstream flow of blood.

Any of the cannulae described herein may be made from various materials to improve their viability in long-term treatment applications. For example, it is preferred that the biocompatibility of the cannula be improved compared to uncoated cannulae to prevent adverse reactions such as compliment activation and the like. To prevent such side effects, the interior lumens of the cannulae can be coated with biocompatible materials. Also known in the art are anti-bacterial coatings. Such coatings may be very useful on the outer surface of the cannula. This is especially true at or about where the cannula enters the patient's skin. At such a location, the patient is vulnerable to introduction of bacteria into the body cavity. Anti-bacterial coatings can reduce the likelihood of infection and thus improve the viability of long-term treatments.

In one application, a cannula may be integrated into a heart assist system. The heart assist system may be configured in any number of ways. Various heart assist systems have been described above. In addition, as shown in to FIG. 20, in one embodiment such a system comprises the cannula 700, an inflow conduit 776, an outflow conduit 780 and a pump 784. One end of the outflow conduit 780 may be connected to the proximal end of the first lumen 716, while the other end is connected to the inlet of the pump 784. One end of the inflow conduit 776 may be connected to the proximal end of the second lumen 720, while the other end is connected to the outlet of the pump 784. This results in a flow from the first distal end 712 to the second distal end 724. Of course, the flow direction may be reversed using the same cannula, resulting in a flow from the second distal end 724 to the first distal end 712. In that case, the outflow conduit 780 is connected to the proximal end of the second lumen 720 and the inflow conduit 776 is connected to the proximal end of the first lumen 716.

Referring to FIG. 20, the cannula 700 may be applied to a patient in an arterial-arterial fashion, e.g., with the cannula 700 inserted into the femoral artery 788 of the patient 792. Where provided, the radiopaque marker 744 is used to track the insertion of the cannula 700 so that the cannula may be positioned at a desired site within the patient's vascular system. As mentioned above, markings 748 near the proximal end 708 could also be used to locate the distal end or ends of the cannula 700. In one application, the first distal end 712 may advance up to the thoracic aorta or even further.

In operation, the pump draws blood from the patient's vascular system in the area near the distal end 724 and into the second lumen 720. The blood is further drawn into the lumen of the inflow conduit 780 and into the pump 784. The pump 784 then expels the blood into the lumen of the outflow conduit 776. The lumen of the outflow conduit 776 carries the blood into the second lumen 716 of the cannula 700 and back into the patient's vascular system in the area near the distal end 712.

According to one method of treating a patient using an extracardiac heart assist system, the cannula system is inserted into the vasculature of a patient and selectively actuated to enhance blood flow past the cannula. As described in greater detail below, with reference to embodiments illustrated in FIGS. 21-26, the additional lumen, the inflatable members, and/or the sleeve and apertures selectively provide blood flow to the patient's vasculature downstream of where the cannulae reside in the vasculature to maintain or enhance perfusion of blood, e.g., by active or by passive perfusion.

Referring to FIGS. 21 and 22, the perfusion lumen 732 of the embodiment shown in FIG. 17 is located entirely within the vessel 788 when the cannula 700 is inserted into the patient. In one embodiment, the lumen 732 can be selectively actuated by inflating the balloon 704 with the use of a syringe or other inflation means, such as, for example, those used for angioplasty balloons. The lumen 732 provides a pathway for blood flow to tissue downstream of the cannula so that the cannula 700 may maintain or increase the flow of blood to downstream tissue. In one embodiment, the lumen 732 is advantageously configured to extend the entire length of the potentially occluded portion of the vessel. For example, as shown in FIG. 21, the perfusion lumen 732 extends from a location distal of the distal end 724 at least to the vascular insertion site. This enables blood to enter the lumen 732 upstream of the distal end 724 and to be conveyed past the occluded region of the vessel to a location where the blood exiting the lumen 732 can flow substantially uninhibited beyond the insertion site. The lumen 732, thus, provides passive perfusion. If desired, apertures may be included in one of the other two lumens 716, 720 to supplement passive perfusion with active perfusion.

Referring to FIGS. 23 and 24, the inflatable members or balloons 804 of the embodiment shown in FIG. 18 are located entirely within the vessel 888 when the cannula 800 is inserted into the patient. In one embodiment, the balloons 804 can be selectively actuated by inflating the balloons 804 with the use of a syringe or other inflation means, as described above. Spaces 866 created alongside the balloons 804 provide pathways for blood to flow to tissue downstream of the cannula 800 providing passive perfusion. If desired, apertures may be included in one of the other two lumens 816, 820 to supplement passive perfusion with active perfusion.

Referring to FIGS. 25 and 26, the cannula system 900, as described with reference to FIG. 19, comprises features that will maintain or increase the blood flow to downstream tissue when the cannula is inserted into the patient. The perfusion cannula system 900 can be selectively actuated by moving the sleeve 972 relative the apertures 968 to uncover the apertures 968. In one embodiment, selectively actuating the cannula system 900 comprises twisting the cannula system within the vasculature to expose the apertures 968. The apertures 968 provide for fluid communication between at least one lumen 916 or 920 and the patient's blood vessel 988. The apertures 968 thus provide active perfusion of the downstream tissues.

Although the foregoing invention has been described in terms of certain preferred embodiments, other embodiments will be apparent to those of ordinary skill in the art. Additionally, other combinations, omissions, substitutions and modification will be apparent to the skilled artisan, in view of the disclosure herein. Accordingly, the present invention is not intended to be limited by the recitation of the preferred embodiments, but is instead to be defined by reference to the appended claims.

1. A perfusion cannula system for directing blood through the vasculature of a patient, comprising:
a cannula body comprising a proximal end, a distal end, and at least one lumen extending therebetween;
a balloon located on an exterior surface of the cannula body; and
means for deploying the balloon within the vasculature
whereby space may be provided between a vessel wall and the cannula body when the cannula body resides within the patient to permit blood flow past the cannula body.

2. The cannula system of claim 1, wherein the balloon defines a perfusion lumen when deployed.

3. The cannula system of claim 1, wherein the balloon comprises a first balloon and further comprising at least a second balloon spaced radially from the first balloon.

4. The cannula system of claim 1, further comprising a second lumen.

5. The cannula system of claim 1, wherein the deploying means comprises an inflation lumen.

6. A perfusion cannula system for directing blood through the vasculature of a patient, comprising:
a cannula body comprising a proximal end, a distal end, and at least one lumen extending therebetween; and
means for creating space around the cannula body within the vasculature to permit blood flow past the cannula body.

7. The cannula system of claim 6, wherein the space creating means is coupled with the cannula body.

8. The cannula system of claim 6, wherein the space creating means is integral with the cannula body.

9. The cannula system of claim 6, wherein the space creating means comprises a collapsible element.

10. The cannula system of claim 6, wherein the space creating means comprises an expandable element.

11. A perfusion system for directing blood through the vasculature of a patient, comprising a multilumen cannula and a plurality of radially spaced balloons configured to be selectively inflated while residing with the vasculature to create space around the cannula within the vasculature to permit blood flow past the cannula.

12. The perfusion system of claim 11, wherein the balloons are integrally formed with the cannula.

13. The perfusion system of claim 11, wherein the cannula comprises inflation lumens.

14. A perfusion cannula system, comprising:
a cannula comprising a cannula body defining at least one lumen extending between a proximal end and a distal end, said cannula body having an aperture formed therein in fluid communication with said lumen; and
a sleeve carried by the cannula and configured to be moveable relative to the aperture to selectively cover and uncover the aperture as desired.

15. The cannula system of claim 14, where the sleeve is carried on the outside of the cannula body.

16. The perfusion cannula system of claim 14, wherein the sleeve is configured to move radially with respect to the cannula body.

17. The perfusion cannula system of claim 14, wherein the sleeve is configured to move longitudinally distally and proximally with respect to the cannula body.

18. The perfusion cannula system of claim 14, further comprising at least one additional aperture

19. The perfusion cannula system of claim 14, further comprising a second lumen.

20. A perfusion cannula system comprising:
a cannula body comprising a proximal end, a distal end, at least one lumen extending therebetween, and a means for enhancing blood flow past the cannula when the cannula body resides within the patient.

21. The cannula system of claim 20, wherein the enhancing means is capable of selectively enhancing blood flow past the cannula.

22. The cannula system of claim 20, wherein the enhancing means comprises at least one balloon.

23. The cannula system of claim 20, wherein the enhancing means comprises at least one balloon defining a perfusion lumen.

24. The cannula system of claim 20, wherein the enhancing means of the cannula body comprises:
at least one aperture defined in the cannula body in fluid communication with said lumen; and
a sleeve carried by the cannula and configured to be moveable relative to the aperture to selectively cover and uncover the aperture as desired.

25. An extracardiac heart assist system, comprising:
a pump having an inlet and an outlet;
an inflow conduit coupled with the inlet;
an outflow conduit coupled with the outlet; and
an intravascular conduit having a proximal end, a distal end, at least one lumen extending therebetween, and a means for selectively enhancing blood flow past the cannula when the cannula resides within the patient, the intravascular conduit configured to provide fluid communication between the vasculature of a patient and at least one of the inflow conduit and the outflow conduit..

26. The extracardiac heart assist system of claim 25, wherein the intravascular conduit is a first conduit configured to couple the inflow conduit to the vasculature of the patient at a first location, and further comprising a second intravascular conduit configured to couple the outflow conduit to the vasculature of the patient at a second location.

27. The extracardiac heart assist system of claim 25, wherein the intravascular conduit is configured to couple the inflow conduit and the outflow conduit to the vasculature of the patient at a single location.

28. The extracardiac heart assist system of claim 25, wherein the intravascular conduit further comprises a plurality of lumens extending between the proximal end and the distal end.

29. The extracardiac heart assist system of claim 25, wherein the pump is configured for insertion within the patient.

30. The extracardiac heart assist system of claim 25, wherein the pump is configured for use outside the patient.

31. The extracardiac heart assist system of claim 25, wherein the pump is configured to pump blood through the patient at subcardiac volumetric rates, the pump having an average flow rate that, during normal operation thereof, is substantially below that of the patient's heart when healthy.

32. The extracardiac heart assist system of claim 25, further comprising a reservoir coupled with the inflow conduit or the outflow conduit.

33. The extracardiac heart assist system of claim 25, wherein the enhancing means comprises at least one balloon coupled to the cannula body.

34. The extracardiac heart assist system of claim 25, wherein the enhancing means of the cannula body comprises:
at least one aperture defined in the cannula body in fluid communication with said lumen; and
a sleeve carried by the cannula and configured to be moveable relative to the aperture to selectively cover and uncover the aperture as desired.

35. A method of treating a patient using an extracardiac heart assist system, comprising:
inserting a cannula system into the vasculature of a patient, wherein the cannula system is actuatable to enhance blood flow past the cannula when the cannula resides in the vasculature of the patient;
selectively actuating the cannula system, whereby blood flow past the cannula is enhanced.

36. The method of claim 35, wherein the cannula system comprises a balloon and wherein selectively actuating the cannula system comprises selectively inflating the balloon.

37. The method of claim 35, wherein the cannula system comprises an aperture formed in a cannula wall and a sleeve disposed about the cannula wall covering the aperture and wherein selectively actuating the cannula system comprises selectively moving the sleeve relative the aperture to uncover the aperture.

38. The method of claim 35, wherein selectively actuating the cannula system comprises twisting the cannula system within the vasculature.

39. The method of claim 35, wherein selectively actuating the cannula system comprises expanding or contracting a portion of the cannula system to provide a blood carrying space between a vessel wall and the cannula system when applied to the patient.
